(19) United States
(12) Patent Application Publication
(10) Pub. No.: US 2025/0387333 A1
(43) Pub. Date: Dec. 25, 2025

(54) CARBON NANOPARTICLE, METHOD FOR PRODUCING THE SAME AND USE OF THE SAME

(71) Applicant: CHANG GUNG UNIVERSITY, Taoyuan City (TW)

(72) Inventors: Jui-Yang LAI, Taoyuan City (TW); Huan-Tsung CHANG, Taoyuan City (TW); Chih-Ching HUANG, Taoyuan City (TW); Han-Jia LIN, Taoyuan City (TW); Hong-Jyuan JIAN, Taoyuan City (TW)

(21) Appl. No.: 18/987,531

(22) Filed: Dec. 19, 2024

(30) Foreign Application Priority Data
Jun. 20, 2024 (TW) 113122831

(51) Int. Cl. A61K 9/14; A61K 9/00; A61K 9/08; A61K 33/44; C01B 32/15

(52) U.S. Cl. CPC A61K 9/14; A61K 9/0048; A61K 9/08; A61K 33/44; C01B 32/15; C01P 2002/82; C01P 2002/84; C01P 2004/03; C01P 2004/62; C01P 2004/64; C01P 2006/40

(57) ABSTRACT

A carbon nanoparticle, which is produced by the step of subjecting sodium alginate and a diamine to a pyrolysis treatment at a temperature ranging from 160° C. to 200° C. The diamine is a C₄-C₁₀ linear aliphatic diamine. A method for alleviating ocular angiogenesis using the carbon nanoparticle and a method for producing the carbon nanoparticle are also provided.

### CROSS-REFERENCE TO RELATED APPLICATION

This application claims priority to Taiwanese Invention Patent Application No. 113122831, filed on Jun. 20, 2024, the entire disclosure of which is incorporated by reference herein.

### FIELD

The disclosure relates to a carbon nanoparticle and a method for producing the same. The disclosure also relates to a method for alleviating ocular angiogenesis using the carbon nanoparticle.

### BACKGROUND

Ocular angiogenesis refers to the formation of new blood vessels from existing vascular tree, which may lead to visual impairment or even irreversible blindness, and choroidal neovascularization (CNV) is the most common type of ocular angiogenesis, among others. Ocular angiogenesis has been reported to be a cause of vision loss in many ocular disorders, including age-related macular degeneration (AMD), diabetic retinopathy, multifocal choroiditis, polypoidal choroidal vasculopathy (PCV), and so forth.

Methods used clinically for treatment of the ocular angiogenesis may include laser photocoagulation, transpupillary thermotherapy (TTT), photodynamic therapy (PDT), and anti-angiogenesis therapy, among which the anti-angiogenesis therapy is most widely employed, and is achieved by virtue of intravitreal injection of a medication containing an anti-vascular endothelial growth factor (VEGF) to inhibit angiogenesis. However, completion of the anti-angiogenesis therapy usually requires several times of injections, which not only results in a high medication cost but also easily causes serious side effects and adverse effects, such as endophthalmitis, retinal detachment, high intraocular pressure, etc., in patients.

Therefore, there is still a need to develop a medication which can exhibit an excellent effect of anti-ocular angiogenesis at a low administration frequency without causing undesirable side effects.

### SUMMARY

Accordingly, in a first aspect, the present disclosure provides a carbon nanoparticle, which can alleviate at least one of the drawbacks of the prior art. The carbon nanoparticle is produced by the step of subjecting sodium alginate and a diamine to a pyrolysis treatment at a temperature ranging from 160° C. to 200° C. The diamine is a C₄-C₁₀ linear aliphatic diamine.

In a second aspect, the present disclosure provide a method for alleviating ocular angiogenesis, which can alleviate at least one of the drawbacks of the prior art. The method includes administering to a subject in need thereof a pharmaceutical composition containing the aforesaid carbon nanoparticle.

In a third aspect, the present disclosure provides a method for producing a carbon nanoparticle, which can alleviate at least one of the drawbacks of the prior art. The method includes the step of subjecting sodium alginate and a diamine to a pyrolysis treatment at a temperature ranging from 160° C. to 200° C. The diamine is a C₄-C₁₀ linear aliphatic diamine.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the disclosure will become apparent in the following detailed description of the embodiment(s) with reference to the accompanying drawings. It is noted that various features may not be drawn to scale.

FIG. 1 shows the ultraviolet-visible (UV-Vis) absorption spectra of solutions containing carbon nanoparticles of experimental groups 1 to 4 prepared in Example 1, infra.

FIG. 2 shows the transmission electron microscope images of SA-CNP, SA/DAO-CNP1, SA/DAO-CNP2 and SA/DAO-CNP3.

FIG. 3 shows the Fourier transform infrared (FTIR) spectroscopy spectra of SA-CNP, SA/DAO-CNP1, SA/DAO-CNP2 and SA/DAO-CNP3.

FIG. 4 shows the migration distance determined in each group of Example 3, infra, in which the symbols “*” and “***” respectively represent p<0.05 and p<0.001 (compared with the pathological control group).

FIG. 5 shows the tube formation percentage determined in each group of Example 3, infra, in which the symbols “*”, “**”, and “***” respectively represent p<0.05, p<0.01, and p<0.001 (compared with the pathological control group).

FIG. 6 shows the relative blood vessel area determined in each group of Example 4, infra, in which the symbols “*” and “***” respectively represent p<0.05 and p<0.001 (compared with the pathological control group).

FIG. 7 shows the result of hematoxylin-eosin staining of posterior segment tissue sections of the rabbits in each group of Example 4, infra, in which the green arrows indicate the formation of blood vessels, and the “IPL”, the “INL”, the “OPL”, the “ONL”, and the “RPE” respectively represent inner plexiform layer, inner nuclear layer, outer plexiform layer, outer nuclear layer, and retinal pigment epithelium.

FIG. 8 shows the relative fluorescence intensity determined in each group of Example 4, infra, in which the symbols “*” and “***” respectively represent p<0.05 and p<0.005 (compared with the pathological control group).

FIG. 9 shows the cell proliferation percentage determined in each group of Example 5, infra.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

By conducting research, the applicant unexpectedly discovered that carbon nanoparticles can be produced by subjecting sodium alginate (SA) and a diamine, which is a C₄-C₁₀ linear aliphatic diamine, to a pyrolysis treatment at a temperature ranging from 160° C. to 200° C., and also found, through both in vitro and in vivo tests, that the carbon nanoparticles could effectively inhibit vascular endothelial growth factor (VEGF)-induced cell migration, VEFG-induced tube formation, and VEGF/basic fibroblast growth factor (bFGF)-induced choroidal neovascularization, and alleviate tissue damage occurring in the posterior segment of an eye of a white rabbit without causing a severe side effect thereto, and hence the carbon nanoparticles were expected to be effective in anti-ocular angiogenesis.

Therefore, the present disclosure provides a carbon nanoparticle, which is produced by the step of subjecting sodium alginate (SA) and a diamine to a pyrolysis treatment at a temperature ranging from 160° C. to 200° C. The diamine is a C₄-C₁₀ linear aliphatic diamine.

In certain embodiments, the temperature may be 180° C.

In certain embodiments, a weight ratio of the sodium alginate to the diamine may range from 1:0.1 to 1:0.5. In an exemplary embodiment, the weight ratio of the sodium alginate to the diamine is 1:0.1. In another exemplary embodiment, the weight ratio of the sodium alginate to the diamine is 1:0.5.

According to the present disclosure, the carbon nanoparticle may have a particle size ranging from 20.0 nm to 461.2 nm. In certain embodiments, the carbon nanoparticle has the particle size ranging from 29.8 nm to 461.2 nm. In an exemplary embodiment, the carbon nanoparticle has the particle size ranging from 179.6 nm to 461.2 nm.

According to the present disclosure, the carbon nanoparticle may have a surface zeta (ζ) potential ranging from −20.0 mV to −39.0 mV. In certain embodiments, the carbon nanoparticle has the surface zeta (ζ) potential ranging from −28.4 mV to −36.5 mV. In an exemplary embodiment, the carbon nanoparticle has the surface zeta (ζ) potential ranging from −31 mV to −33.4 mV.

According to the present disclosure, the carbon nanoparticle has a double-bond selected from the group consisting of a carbon-carbon double bond (C═C bond), a carbon-oxygen double bond (C═O bond), carbon-nitrogen double bond (C═N bond), and combinations thereof.

The present disclosure also provides a method for producing a carbon nanoparticle, which includes the step of subjecting sodium alginate and a diamine to a pyrolysis treatment at a temperature ranging from 160° C. to 200° C. The diamine is a C₄-C₁₀ linear aliphatic diamine. In certain embodiments, the diamine may be the Ca linear aliphatic diamine.

In some embodiments, the diamine may be selected from the group consisting of 1,4-diaminobutane (DAB), 1,6-diaminohexane (DAH), 1,8-diaminooctane (DAO), 1,10-diaminodecane (DAD), and combinations thereof.

In certain embodiments, the temperature may be 180° C.

In certain embodiments, a weight ratio of the sodium alginate to the diamine may range from 1:0.1 to 1:0.5. In an exemplary embodiment, the weight ratio of the sodium alginate to the diamine is 1:0.1. In another exemplary embodiment, the weight ratio of the sodium alginate to the diamine is 1:0.5.

According to the present disclosure, the pyrolysis treatment may be carried out using techniques well known to those skilled in the art. In this regard, reference may be made to, for example, TW I773949 B and TW I815436 B.

It can be understood that, in order to achieve the best pyrolysis effect, the conditions for carrying out the pyrolysis treatment may be varied according to actual factors such as the weight ratio of the sodium alginate to the diamine, and selection of the conditions for carrying out the pyrolysis treatment is within the expertise and routine skills of those skilled in the art.

According to the present disclosure, the pyrolysis treatment is carried out for a time period ranging from 2 hours to 6 hours. In some embodiments, the pyrolysis treatment may be carried out at the temperature of 180° C. for 3 hours.

According to the present disclosure, after the pyrolysis treatment, the sodium alginate and the diamine that are unreacted can be removed using techniques well known to those skilled in the art. In certain embodiments, the sodium alginate and the diamine that are unreacted may be removed by dialysis.

The present disclosure further provides a method for alleviating ocular angiogenesis, which includes administering to a subject in need thereof a pharmaceutical composition containing the aforesaid carbon nanoparticles.

As used herein, the term “administering” and “administration” can be interchangeably used, and mean introducing, providing or delivering a pre-determined active ingredient (e.g., the above-mentioned pharmaceutical composition) to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the pharmaceutical composition may be formulated into a dosage form suitable for intraocular administration or topical ophthalmic administration using technology well known to those skilled in the art.

According to the present disclosure, the dosage form suitable for intraocular administration includes, but is not limited to, an injection, e.g., a sterile aqueous solution, a dispersion or an emulsion.

The pharmaceutical composition according to the present disclosure may be administered via one of the following routes: subtenon injection, intravitreal injection, intracameral injection, intra-retinal injection, subretinal injection, and suprachoroidal injection.

According to the present disclosure, the dosage form suitable for topical ophthalmic administration includes, but is not limited to, drops, emulsions, gels, ointments, creams, sprays, micelles, and suspensions.

According to the present disclosure, the pharmaceutical composition may further include a pharmaceutically acceptable carrier widely employed in the art of drug-manufacturing. For instance, the pharmaceutically acceptable carrier may include one or more of the following agents: solvents (e.g., a sterile water), buffers (e.g., an ophthalmic balanced salt solution, phosphate buffered saline (PBS), Ringer's solution and Hank's solution), emulsifiers, suspending agents, decomposers, pH adjusting agents, stabilizing agents, chelating agents, preservatives, diluents, absorption delaying agents, liposomes, lubricants, and the like. The choice and amount of the aforesaid agents are within the expertise and routine skills of those skilled in the art.

The dose and frequency of administration of the pharmaceutical composition of the present disclosure may vary depending on the following factors: the severity of the illness or disorder to be treated, routes of administration, and age, physical condition and response of the subject to be treated. In general, the pharmaceutical composition may be administered in a single dose or in several doses. In certain embodiments, the pharmaceutical composition of the present disclosure may be administered in the single dose.

The present disclosure will be further described by way of the following examples. However, it should be understood that the following examples are intended solely for the purpose of illustration and should not be construed as limiting the present disclosure in practice.

### EXAMPLES

### General Experimental Materials:

1. The sodium alginate (SA) and the diamines, as shown in Table 1 below, used in the following examples were all purchased from Sigma-Aldrich.

TABLE 1
Diamines

1,4-diaminobutane (DAB)
1,6-diaminohexane (DAH)
1,8-diaminooctane (DAO)
1,10-diaminodecane (DAD)

### General Procedures:

### 1. Statistical Analysis:

The experimental data of all the test groups are expressed as mean±standard deviation (SD), and were analyzed using one-way analysis of variance (one-way ANOVA) followed by Newman-Keuls post hoc test, so as to evaluate the differences between the groups. Statistical significance is indicated by p<0.05.

### Example 1. Preparation of Carbon Nanoparticles According to the Present Disclosure

The sodium alginate (SA) was divided into four groups, namely, experimental groups 1 to 4 (50 mg of the SA in each group). A corresponding amount of the 1,8-diaminooctane (DAO, serving as a diamine) as shown in Table 2 below was then added to each group of the SA, followed by mixing with 4 mL of ultrapure water, so as to obtain a mixture. Thereafter, the mixture was placed in a muffle furnace, and then subjected to a pyrolysis treatment at 180° C. for 3 hours, so as to allow a carbonization reaction to proceed, thereby forming a pyrolyzed product. Next, the pyrolyzed product of each group was cooled to room temperature, and then 5 mL of deionized water was added thereto, followed by ultra-sonication for 1 hour. After centrifugation at 500 g for 30 minutes, the supernatant was collected, thereby obtaining a solution containing carbon nanoparticles.

TABLE 2

| Group | Amount of SA (mg) | Amount of DAO (mg) |
|---|---|---|
| Experimental group 1 | 50 | — |
| Experimental group 2 | 50 | 5 |
| Experimental group 3 | 50 | 12.5 |
| Experimental group 4 | 50 | 25 |

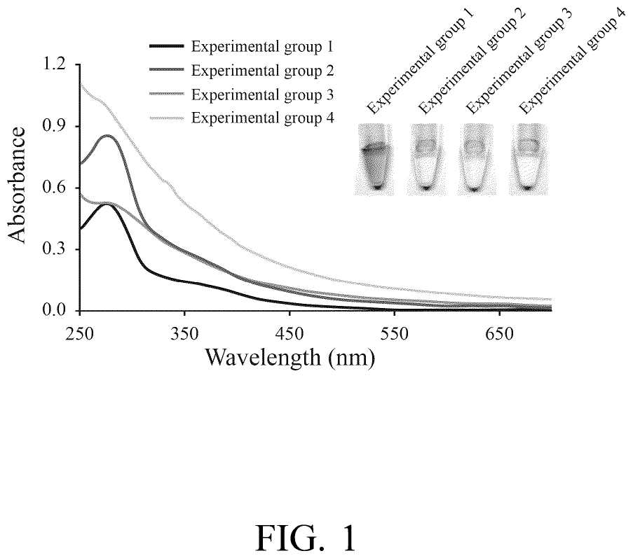

Subsequently, the solution containing the carbon nanoparticles was diluted 10-fold with deionized water, and then subjected to measurement of ultraviolet-visible (UV-Vis) absorption using a monochromatic microplate spectrophotometer (Synergy 4 Multi-Mode, Biotek Instruments, Winooski, VT, USA), thereby obtaining an UV-Vis absorption spectrum of the solution of each of the experimental groups 1 to 4. The results are shown in FIG. 1.

Referring to FIG. 1, the solution in each of the experimental groups 1 to 4 had a broad absorption band at a wavelength of approximately 270 nm, indicating that a π→π* conversion occurred, resulting in the formation of carbon-carbon double bonds (C═C bonds). Moreover, an absorption band at a wavelength ranging from 300 nm to 420 nm could also be found, indicating that an n→π* conversion occurred, resulting in the formation of carbon-oxygen double bonds (C═O bonds) and carbon-nitrogen double bonds (C═N bonds). These results demonstrate that by virtue of subjecting a combination of the SA and the DAO to the pyrolysis treatment at 180° C., the carbon nanoparticles can be successfully generated.

### Example 2. Analysis of Physical and Chemical Properties of Carbon Nanoparticles According to the Present Disclosure

### A. Morphological Analysis

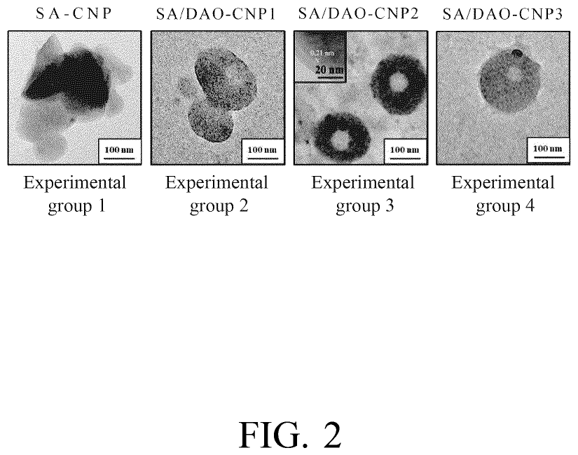

First, the solution of each of the experimental groups 1 to 4 obtained in Example 1 was subjected to dialysis using a dialysis membrane with a molecular weight cut-off value of 3 kDa and deionized water for five times, with the first four times of dialysis being performed for 1 hour each time, and the last time of dialysis being performed overnight, so as to remove the SA and the DAO that were unreacted, thereby obtaining a dialysate containing the carbon nanoparticles. The dialysates thus obtained served as test samples of the experimental groups 1 to 4. Subsequently, the test sample of each of the experimental groups 1 to 4 was subjected to morphological analysis using a Tecnai 20 G2 S-Twin transmission electron microscope (Philips/FEI, Hillsboro, OR, USA). The results are shown in FIG. 2.

Referring to FIG. 2, the carbon nanoparticles in the experimental group 1 were irregular-shaped carbon nanoparticles (irregular-shaped CNP; hereinafter abbreviated as “SA-CNP”), while the carbon nanoparticles in all of the experimental groups 2 to 4 were donut-shaped carbon nanoparticles (donut-shaped CNP; hereinafter abbreviated as “SA/DAO-CNP1”, “SA/DAO-CNP2” and “SA/DAO-CNP3”, respectively). These results show that by virtue of crosslinking, the SA and the DAO can form amide bonds and network structures, and through partial carbonization of the SA and the DAO, the carbon nanoparticles can be generated.

### B. Fourier Transform Infrared (FTIR) Spectroscopy Analysis

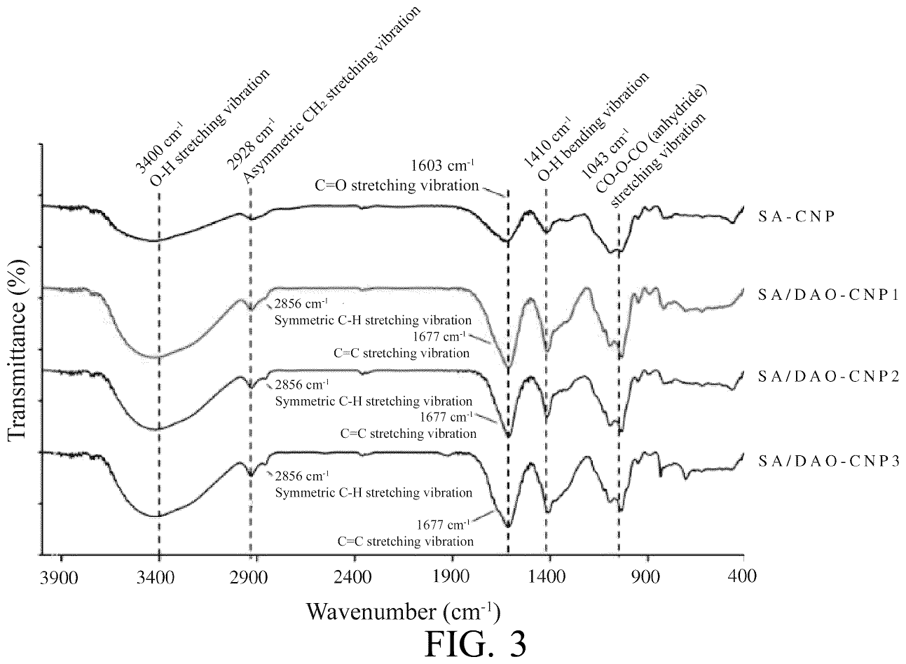

A suitable amount of the dialysate containing a respective one of SA-CNP, SA/DAO-CNP1, SA/DAO-CNP2 and SA/DAO-CNP3 obtained in Section A of this example was subjected to lyophilization, so as to obtain a lyophilized powder. Thereafter, the lyophilized powder was mixed with potassium bromide in a weight ratio of 1:99, and the mixture thus obtained served as a test sample. After that, the test sample was subjected to FTIR analysis using an FT-730 ATR/FTIR spectrometer (Horiba, Japan). The results are shown in FIG. 3.

Referring to FIG. 3, each of the SA-CNP, the SA/DAO-CNP1, the SA/DAO-CNP2, and the SA/DAO-CNP3 had characteristic peaks at wavenumbers of 1043 cm⁻¹ (anhydride (CO—O—CO) stretching vibration), 1410 cm⁻¹ (0-H bending vibration), 1603 cm⁻¹ (C═O stretching vibration), 2928 cm⁻¹ (asymmetric CH₂ stretching vibration), and 3400 cm⁻¹ (O—H stretching vibration). In particular, each of the SA/DAO-CNP1, the SA/DAO-CNP2, and the SA/DAO-CNP3 also had characteristic peaks at wavenumbers of 1677 cm⁻¹ (C═C stretching vibration) and 2856 cm⁻¹ (symmetric C—H stretching vibration). However, the SA-CNP did not show such characteristic peaks at the wavenumbers of 1677 cm⁻¹ and 2856 cm⁻¹. These results indicate that the bonding and structural properties of the carbon nanoparticles (i.e., the SA/DAO-CNP1, the SA/DAO-CNP2, and the SA/DAO-CNP3) prepared using both the SA and the DAO were obviously different from those of the carbon nanoparticles (i.e., the SA-CNP) prepared using solely the SA.

### C. Surface Zeta (ζ) Potential Analysis

A suitable amount of the solution containing a respective one of SA-CNP, SA/DAO-CNP1, SA/DAO-CNP2 and SA/DAO-CNP3 obtained in Example 1 was dissolved in 5 mM of phosphate buffer (PB) (pH 7.4). Each of the resultant mixture was then subjected to surface zeta (ζ) potential analysis using a Zetasizer Nano ZS analyzer (Malvern Instruments, Worcestershire, UK). The results are shown in Table 3 below.

TABLE 3

| Carbon nanoparticles | Surface zeta (ζ) potential (mV) |
|---|---|
| SA-CNP | −44.5 ± 5.4 |
| SA/DAO-CNP1 | −35.3 ± 1.2 |
| SA/DAO-CNP2 | −32.2 ± 1.2 |
| SA/DAO-CNP3 | −31.9 ± 3.5 |

Referring to FIG. 3, the carbon nanoparticles (i.e., the SA/DAO-CNP1, the SA/DAO-CNP2, and the SA/DAO-CNP3) prepared using both the SA and the DAO had surface zeta (ζ) potentials approximately ranging from −28.4 mV to −36.5 mV, which were significantly different from the surface zeta (ζ) potential (−44.5±5.4) of the carbon nanoparticles (i.e., the SA-CNP) prepared using solely the SA.

### Example 3. Evaluation of the Efficacy of Carbon Nanoparticles According to the Present Disclosure in Alleviating Pathologic Ocular Angiogenesis In Vitro

In this example, the applicant selected human umbilical vein endothelial cells (HUVECs) and utilized cell migration assay and tube formation assay to evaluate the anti-angiogenic effects of the carbon nanoparticles according to the disclosure.

### Experimental Materials:

### 1. Source and Cultivation of HUVECs

The HUVECs used in this example were purchased from Lifeline Cell Technology, LLC. The HUVECs were cultivated in a 10-cm Petri dish containing a VasucLife® EnGS endothelial cell growth medium (hereinafter abbreviated as “ECG medium”) (Lifeline Cell Technology, LLC), and then incubated in an incubator with culture conditions set at 37° C. and 5% CO₂. Medium change was performed every two to three days. Cell passage was performed when the cultured cells reached 80% of confluence.

### Experimental Procedures and Results:

### A. Cell Migration Assay

The HUVECs prepared in Section 1 of Experimental Materials of this example were divided into six groups, including a normal control group, a pathological control group, a comparative group, and three experimental groups (i.e., experimental groups 1 to 3). Next, multiple Culture-Insert 2 Well (ibidi GmbH) were placed in respective wells of a 24-well culture plate, where each well contained a suitable amount of ECG medium, and then each group of the HUVECs was seeded at a concentration of 1.3×10⁴ cells/well in a respective one of the multiple Culture-Insert 2 Well, followed by cultivation in an incubator with culture conditions set at 37° C., 5% CO₂ for 12 hours. After that, the Culture-Insert 2 Well in each well of the 24-well culture plate was removed, so as to expose a gap without the HUVECs attached thereto. Next, the ECG medium in each group was replaced with a fresh ECG medium supplemented with 2% of fetal bovine serum (FBS). The cell culture in each of the experimental groups 1 to 3 and the comparative group was then treated with a suitable amount of vascular endothelial growth factor-165 (VEGF-165) (R&D systems, Cat. No. 293-VE-050/CF) and a corresponding type of the carbon nanoparticles as shown in Table 4 below. In addition, the cell culture in the pathological control group was only treated with VEGF-165, and the cell culture in the normal control group received no treatment.

TABLE 4

| | Treating agent | |
|---|---|---|
| | VEGF-165 (final | Carbon nanoparticles |
| | concentration: | (final concentration: |
| Group | 250 pM) | 25 μg/mL) |
| Normal control group | − | — |
| Pathological control group | + | — |
| Comparative group | + | SA-CNP |
| Experimental group 1 | + | SA/DAO-CNP1 |
| Experimental group 2 | + | SA/DAO-CNP2 |
| Experimental group 3 | + | SA/DAO-CNP3 |

After cultivation in an incubator with culture conditions set at 37° C., 5% CO₂ for 18 hours, the resultant cell culture in each group was subjected to observation using a phase contrast microscope (Olympus BX 51, Tokyo, Japan) at a magnification of 40×, and to measurement of migration distance (μm).

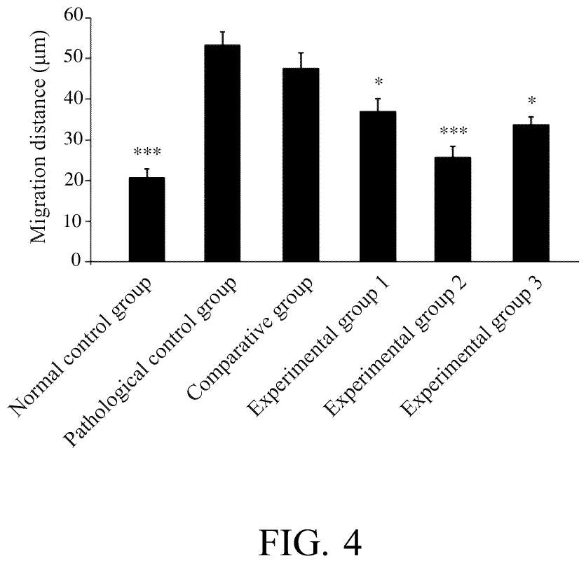

The data thus obtained were analyzed according to the procedures as described in Section 1 of General Experimental Materials. The results are shown in FIG. 4.

Referring to FIG. 4, compared with the normal control group, the migration distance determined in the pathological control group was significantly increased, indicating that VEGF-165 successfully induced the movement of the HUVECs. Moreover, compared with the pathological control group, the migration distance determined in each of the experimental groups 1 to 3 was significantly reduced, in which the migration distance determined in the experimental group 2 had the highest degree of reduction, while the migration distance determined in the comparative group did not show a significant reduction.

### B. Tube Formation Assay

First, 10 mg/mL Matrigel (Corning Inc.) was coated on a polycarbonate membrane (with a pore size of 0.8 μm) of each of Transwell inserts (Corning Inc.), and left to stand at 37° C. for 1 hour. Subsequently, the HUVECs prepared in Section 1 of Experimental Materials of this example were divided into six groups, including a normal control group, a pathological control group, a comparative group, and three experimental groups (i.e., experimental groups 1 to 3). Each group of the HUVECs was seeded at a concentration of 2×10⁴ cells/well in a respective one of the Transwell inserts. Subsequently, the HUVECs in each of the pathological control group, the comparative group, and the three experimental groups were treated with a treating agent according to the method as described in Section A of this example and with reference to Table 4 above, and the HUVECs in the normal control group received no treatment. Thereafter, each of the Transwell inserts was placed in a respective well of a 24-well culture plate containing a suitable amount of ECG medium supplemented with 2% of FBS.

After cultivation in an incubator with culture conditions set at 37° C., 5% CO₂ for 18 hours, the resultant culture in each group was subjected to observation using the phase contrast microscope at a magnification of 40×, so as to observe tubular structures formed on the Matrigel, followed by analysis using ImageJ Imaging Software to determine a tube length. Next, the tube formation percentage (%) of each group was calculated using the following Equation (1):

$$A = ( B / C ) \times 100 \qquad ( 1 )$$

where A=tube formation percentage (%)
   B=tube length of each group (μm)
   C=tube length of the pathological control group (μm)

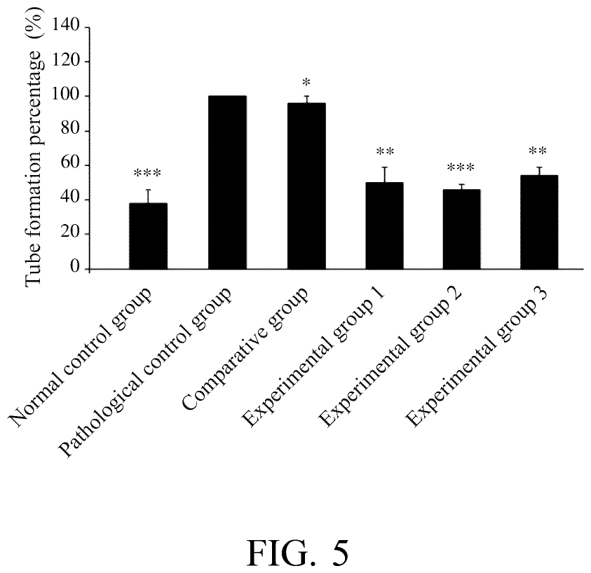

The data thus obtained were analyzed according to the procedures as described in Section 1 of General Experimental Materials. The results are shown in FIG. 5.

Referring to FIG. 5, compared with the normal control group, the tube formation percentage determined in the pathological control group was significantly increased, indicating that the VEGF-165 successfully induced the formation of the tubular structures in the HUVECs. Moreover, compared with the pathological control group, the tube formation percentage measured in each of the experimental groups 1 to 3 was significantly reduced, in which the tube formation percentage measured in the experimental group 2 had the highest degree of reduction, while the tube formation percentage measured in the comparative group only showed a slight degree of reduction.

These results demonstrate that, by virtue of inhibiting VEGF-induced cell migration and VEGF-induced tube formation, all of the SA/DAO-CNP1, the SA/DAO-CNP2, and the SA/DAO-CNP3 exhibited excellent anti-angiogenic effects, particularly the SA/DAO-CNP2. In addition, the SA-CNP barely showed such effect. Accordingly, the carbon nanoparticles that are prepared using different weight ratios of the SA and the DAO are considered to be useful in alleviating pathologic ocular angiogenesis.

### Example 4. Evaluation of Efficacy of Carbon Nanoparticles According to the Present Disclosure in Alleviating Pathologic Ocular Angiogenesis In Vivo

In this example, the applicant selected the SA/DAO-CNP2 to evaluate the anti-angiogenetic effect in vivo, and simultaneously, used the SA-CNP in the following experiments for comparison purpose.

### Experimental Materials:

### 1. Experimental Rabbits

New Zealand white rabbits (16 to 20 weeks old, each having a body weight of approximately 3.0 to 3.5 kg) used in the following experiments were purchased from the National Laboratory Animal Breeding and Research Center. All the experimental rabbits were housed in an animal room with an independent air conditioning system under the following laboratory conditions: an alternating 12-hour light and 12-hour dark cycle, a temperature maintained at a range of 20° C. to 24° C., and a relative humidity maintained at a range of 55% to 65%. Furthermore, water and food were provided ad libitum for all the experimental rabbits. All experimental procedures involving the experimental rabbits were in compliance with the guidelines of the Institutional Animal Care and Use Committee (IACUC) of Chang Gung University and the Association for Research in Vision and Ophthalmology (ARVO).

### 2. Preparation of Ophthalmic Solution Containing Carbon Nanoparticles

A suitable amount of a respective one of the lyophilized powder containing the SA/DAO-CNP2 and the lyophilized powder containing the SA-CNP obtained in Section B of Example 2 was dissolved in a phosphate buffered saline (PBS), thereby obtaining an ophthalmic solution that contains 1 mg/mL SA/DAO-CNP2 (hereinafter abbreviated as “first ophthalmic solution”) and an ophthalmic solution that contains 1 mg/mL SA-CNP (hereinafter abbreviated as “second ophthalmic solution”).

### Experimental Procedures and Results:

### A. Induction of Choroidal Neovascularization

First, 20 μg of VEGF-165 (Cloud-clone crop., RPB696Hu01) was mixed with 15 μg of a basic fibroblast growth factor (bFGF) (PeproTech, Cat. No. 100-18B) and 20 μg of sodium alginate (SA) (10 mg/mL), so as to obtain a hydrogel containing the VEGF-165 and the bFGF.

Subsequently, the New Zealand white rabbits were randomly divided into four groups, including a normal control group, a pathological control group, a comparative group, and an experimental group (n=6 per group). Next, an incision having a length of approximately 3 mm was made at a position that is 2.5 mm from an edge of a cornea of a right eye of a respective one of the rabbits in each of the pathological control group, the comparative group, and the experimental group. Subsequently, the hydrogel was injected into the incision of each rabbit in the pathological control group, the comparative group, and the experimental group using a 2-inch needle, followed by suturing the incision, so as to induce choroidal neovascularization in each rabbit. The rabbits in the normal control group received no treatment.

### B. Administration of Ophthalmic Solution Containing Carbon Nanoparticles

On the 14^{th} day after the injection of the hydrogel, 50 μg of the first ophthalmic solution (with the SA/DAO-CNP2) prepared in Section 2 of the Experimental Materials of this example was injected into a vitreous of the right eye of the respective one of the rabbits in the experimental group, and 50 μg of the second ophthalmic solution (with the SA-CNP) prepared in Section 2 of the Experimental Materials of this example was injected into a vitreous of the right eye of the respective one of the rabbits in the comparative group. The rabbits in each of the normal control group and the pathological control group received no treatment.

### C. Ophthalmoscopy

On the 7^{th} day after the administration of the ophthalmic solution, each rabbit was subjected to observation using an ophthalmoscope (Topcon Optical, Tokyo, Japan), so as to observe the formation of new blood vessels (neovascularization) in a fundus of the right eye, followed by analysis of a blood vessel area (including areas of retinal blood vessels and choroidal blood vessels) utilizing AngioTool software. Thereafter, the relative blood vessel area of each group was calculated using the following Equation (2):

$$D = ( E / F ) \qquad ( 2 )$$

where D=relative blood vessel area
   E=blood vessel area of each group (mm²)
   F=blood vessel area of the normal control group (mm²)

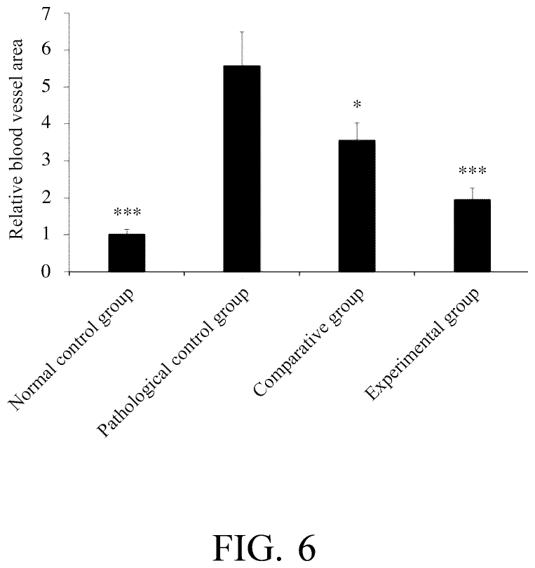

The data thus obtained were analyzed according to the procedures as described in Section 1 of General Experimental Materials. The results are shown in FIG. 6.

Referring to FIG. 6, compared with the normal control group, the relative blood vessel area determined in the pathological control group was significantly increased, indicating that the VEGF-165 and the bFGF successfully induced neovascularization. In addition, compared with the pathological control group, the relative blood vessel area determined in each of the experimental group and the comparative group was significantly reduced, in which the relative blood vessel area determined in the experimental group was significantly lower than that determined in the comparative group.

### D. Hematoxylin-Eosin Staining

After completion of the ophthalmoscopy as described in Section C of this example, the rabbits in each group were sacrificed using CO₂. After that, a posterior segment tissue was cut from the right eye of each rabbit using a scalpel blade, and then was subjected to fixation with 4% paraformaldehyde (in PBS) at room temperature for 60 minutes, followed by embedding with paraffin and slicing, so as to obtain tissue sections each having a thickness of 5 μm.

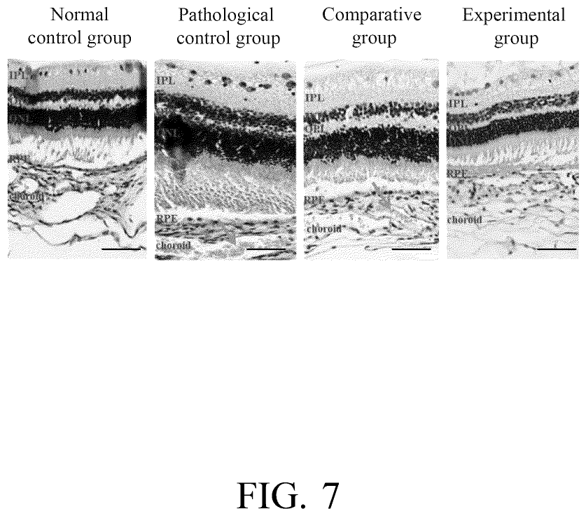

Afterwards, a part of the tissue sections of each group obtained above were subjected to hematoxylin-eosin staining using a staining protocol well known to those skilled in the art. Next, a region of the tissue section, which was randomly selected, was subjected to photography as well as histological observation using an optical microscope (Carl Zeiss, Oberkochen, Germany) at a magnification of 100×. The results are shown in FIG. 7.

Referring to FIG. 7, the occurrence of both hemorrhage and retinal damage (including loss of integrity of an outer nuclear layer (ONL) and loss of integrity of an inner nuclear layer (INL)) could be seen in the posterior segment tissue of the right eye of each rabbit in the pathological control group and the comparative group, and a great amount of blood vessels could also be observed in a choroid (pointed by the green arrow) thereof, indicating that the VEGF-165 and the bFGF successfully induced neovascularization and caused retinal damage in the posterior segment tissue of the right eye. In contrast, the posterior segment tissue of the right eye of each rabbit in the normal control group and the experimental group did not show such hemorrhage and retinal damage.

### E. Immunofluorescence Straining

Another part of the tissue sections of each group obtained in Section D of this example was washed three times with PBS, and was then immersed in a blocking buffer (containing 0.3% Triton-X and 3% FBS dissolved in PBS), so as to allow a reaction to proceed for 1 hour, followed by washing three times with PBS. Thereafter, CD31/PECAM-1 antibody (JC/70A) (Novus Biologicals, Cat. No. NB600-562) was added to the tissue section, followed by incubation at 4° C. for 1 hour. After washing three times with PBS, Goat anti-Mouse IgG (H+L) Cross-Adsorbed Secondary Antibody (Thermo Fisher Scientific, Cat. No. F-2761) conjugated with fluorescein isothiocyanate (FITC) was added to the tissue section, followed by incubation at room temperature for 1 hour. Next, the tissue section was mounted with a mounting medium containing 4,6-diamidino-2-phenylindole (DAPI). The mounted tissue section thus obtained was subjected to observation and photography using a fluorescence microscope (Carl Zeiss, Axiovert 200M) at a magnification of 100×, followed by analysis using ImageJ Imaging Software, so as to obtain a mean fluorescence intensity of each group. Subsequently, the relative fluorescence intensity of each group was calculated using the following Equation (3):

$$G = ( H / I ) \qquad ( 3 )$$

where G=relative fluorescence intensity
   H=mean fluorescence intensity of each group
   I=mean fluorescence intensity of the normal control group

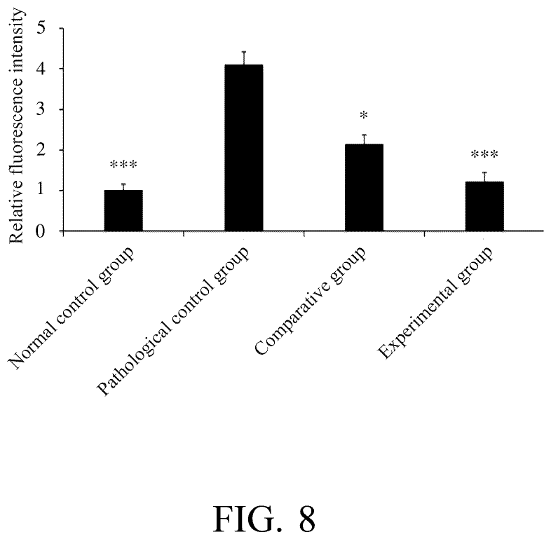

The data thus obtained were analyzed according to the procedures as described in Section 1 of General Experimental Materials. The results are shown in FIG. 8.

Referring to FIG. 8, compared with the normal control group, the relative fluorescence intensity determined in the pathological control group was significantly increased, indicating that the VEGF-165 and the bFGF successfully induced neovascularization. In addition, compared with the pathological control group, the relative fluorescence intensity determined in each of the experimental group and the comparative group was significantly reduced, in which the relative fluorescence intensity determined in the experimental group was significantly lower than that determined in the comparative group, and was even approximate to that determined in the normal control group.

These results demonstrate that the SA/DAO-CNP2 can effectively inhibit choroidal neovascularization induced by co-administration of VEGF-165 and bFGF and alleviate the retinal damage in the posterior segment tissues of the rabbits' eyes. Accordingly, the carbon nanoparticles that are prepared using the SA and the DAO can exhibit an excellent effect in alleviating pathologic ocular angiogenesis in vivo.

### Example 5. Carbon Nanoparticles Prepared Under Different Conditions and Evaluation Efficacy Thereof

### A. Preparation of Carbon Nanoparticles

First, the SA was divided into twelve experimental groups, namely, experimental groups 1 to 12 (50 mg in each group). After that, a solution containing carbon nanoparticles in each group was prepared generally according to the procedures as described in Example 1, except that the types of the diamine and the temperature for conducting the pyrolysis treatment were varied as shown in Table 5 below. The carbon nanoparticles contained in the solutions of the experimental groups 1 to 12 were referred to as “SA/DAB-CNP1 to SA/DAB-CNP3, SA/DAH-CNP1 to SA/DAH-CNP3, SA/DAO-CNP1′ to SA/DAO-CNP3′, and SA/DAD-CNP1 to SA/DAD-CNP3”, respectively.

TABLE 5

| | Diamine | Temperature in pyrolysis |
|---|---|---|
| Group | (12.5 mg per group) | treatment (° C.) |
| Experimental group 1 | 1,4-diaminobutane | 160 |
| Experimental group 2 | (DAB) | 180 |
| Experimental group 3 | | 200 |
| Experimental group 4 | 1,6-diaminohexane | 160 |
| Experimental group 5 | (DAH) | 180 |
| Experimental group 6 | | 200 |
| Experimental group 7 | 1,8-diaminooctane | 160 |
| Experimental group 8 | (DAO) | 180 |
| Experimental group 9 | | 200 |
| Experimental group 10 | 1,10-diaminodecane | 160 |
| Experimental group 11 | (DAD) | 180 |
| Experimental group 12 | | 200 |

### B. Morphological Analysis

First, the solution of each of the experimental groups 1 to 12 obtained in Section A of this example was subjected to dialysis according to the procedures as described in Section A of Example 2, thereby obtaining a dialysate containing the carbon nanoparticles. The dialysate thus obtained served as a test sample of each of the experimental groups 1 to 12. Subsequently, the test sample of each of the experimental groups 1 to 12 was subjected to morphological analysis using the Tecnai 20 G2 S-Twin transmission electron microscope supplemented with a nano measurement software, so as to calculate sizes of the carbon nanoparticles (n=100). The results are shown in Table 6 below.

TABLE 6

| Carbon nanoparticles | Particle size (nm) |
|---|---|
| SA/DAB-CNP1 | 195.8 ± 76.7 |
| SA/DAB-CNP2 | 101.9 ± 72.1 |
| SA/DAB-CNP3 | 183.6 ± 91.9 |
| SA/DAH-CNP1 | 122.1 ± 62.5 |
| SA/DAH-CNP2 | 183.6 ± 80.4 |
| SA/DAH-CNP3 | 336.7 ± 75.1 |
| SA/DAO-CNP1′ | 207.5 ± 103.1 |
| SA/DAO-CNP2′ | 320.4 ± 140.8 |
| SA/DAO-CNP3′ | 336.0 ± 86.5 |
| SA/DAD-CNP1 | 211.4 ± 97.3 |
| SA/DAD-CNP2 | 203.4 ± 20.5 |
| SA/DAD-CNP3 | 204.5 ± 68.1 |

Referring to Table 6, the carbon nanoparticles prepared using the SA and any one of the four diamines (i.e., the DAB, the DAH, the DAO, and the DAD) have particle sizes ranging from approximately 29.8 nm to 461.2 nm.

### C. Assessment of Anti-Angiogenic Effect

The HUVECs prepared in Section 1 of Experimental Materials of Example 3 were divided into fourteen groups, including a normal control group, a pathological control group, and twelve experimental groups (i.e., experimental groups 1 to 12). Each group of the HUVECs were seeded at a concentration of 5×10³ cells/well in a respective well of a 48-well culture plate containing a suitable amount of ECG medium, followed by cultivation in an incubator with culture conditions set at 37° C., 5% CO₂ overnight, so as to allow the HUVECs to attach to the 48-well culture plate. Next, the cell culture in each of the experimental groups 1 to 12 was then treated with a suitable amount of VEGF-165 (R&D systems, Cat. No. 293-VE-050/CF) and a corresponding type of the carbon nanoparticles as shown in Table 7 below. In addition, the cell culture in the pathological control group was only treated with VEGF-165, and the cell culture in the normal control group received no treatment.

TABLE 7

| | Treating agent | |
|---|---|---|
| | VEGF-165 (final | Carbon nanoparticles |
| | concentration: | (final concentration: |
| Group | 250 pM) | 25 μg/mL) |
| Normal control group | − | — |
| Pathological control group | + | — |
| Experimental group 1 | + | SA/DAB-CNP1 |
| Experimental group 2 | + | SA/DAB-CNP2 |
| Experimental group 3 | + | SA/DAB-CNP3 |
| Experimental group 4 | + | SA/DAH-CNP1 |
| Experimental group 5 | + | SA/DAH-CNP2 |
| Experimental group 6 | + | SA/DAH-CNP3 |
| Experimental group 7 | + | SA/DAO-CNP1′ |
| Experimental group 8 | + | SA/DAO-CNP2′ |
| Experimental group 9 | + | SA/DAO-CNP3′ |
| Experimental group 10 | + | SA/DAD-CNP1 |
| Experimental group 11 | + | SA/DAD-CNP2 |
| Experimental group 12 | + | SA/DAD-CNP3 |

After cultivation in an incubator with culture conditions set at 37° C., 5% CO₂ for 72 hours, 200 μL of 3-(4,5-dimethylthiazol-2-yl)-2,5-diphenyltetrazolium bromide (MTT, 0.5 mg/mL) was added to each well, followed by incubation for 30 minutes. Afterwards, the liquid in each well was removed, followed by adding 200 μL of dimethyl sulfoxide (DMSO) and then well mixing, so as to obtain a mixture. Subsequently, the mixture in each well was subjected to determination of absorbance at a wavelength of 570 nm (OD₅₇₀) using a microplate reader (BioTek, Synergy HT).

The cell proliferation percentage (%) of each group was calculated using the following Equation (4):

$$J = ( K / L ) \times 100 \qquad ( 3 )$$

where J=cell proliferation percentage (%)
   K=OD₅₇₀ value determined in each group
   L=OD₅₇₀ value determined in the normal control group

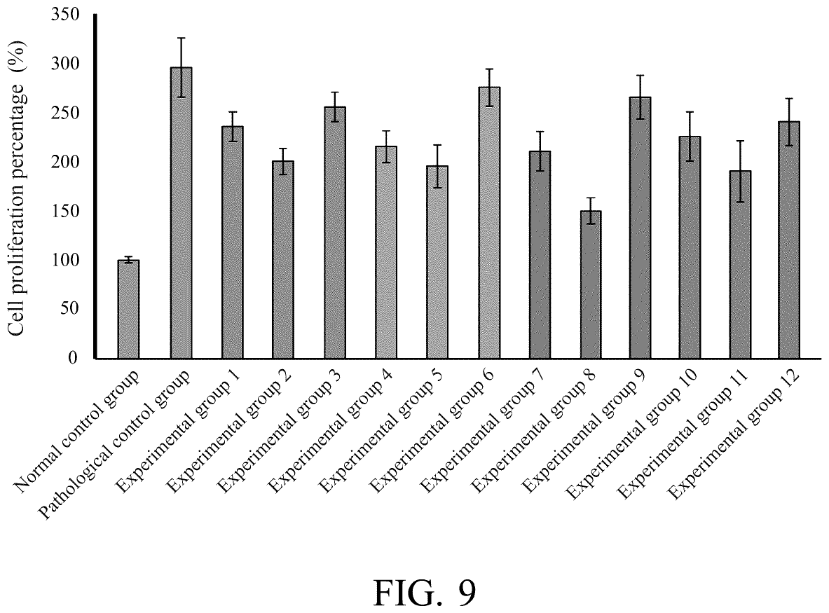

The data thus obtained were analyzed according to the procedures as described in Section 1 of General Experimental Materials. The results are shown in FIG. 9.

Referring to FIG. 9, compared with the normal control group, the cell proliferation percentage determined in the pathological control group was significantly increased, indicating that the VEGF-165 successfully induced the proliferation of the HUVECs. Moreover, compared with the pathological control group, the cell proliferation percentage determined in each of the experimental groups 1 to 12 was significantly reduced, particularly the experimental groups 2, 5, 8, and 11, in which the cell proliferation percentage determined in the experimental group 8 had the most significant decrease.

These results show that, by virtue of subjecting the SA and a C₄-C₁₀ linear aliphatic diamine (such as the DAB, the DAH, the DAO, and the DAD) to the pyrolysis treatment at the temperature ranging from 160° C. to 200° C. (particularly 180° C.), the carbon nanoparticles thus obtained can exhibit excellent anti-angiogenic effects.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A carbon nanoparticle, which is produced by the step of subjecting sodium alginate and a diamine to a pyrolysis treatment at a temperature ranging from 160° C. to 200° C., the diamine being a C₄-C₁₀ linear aliphatic diamine.

2. The carbon nanoparticle as claimed in claim 1, wherein the temperature is 180° C.

3. The carbon nanoparticle as claimed in claim 1, which has a particle size ranging from 20.0 nm to 461.2 nm.

4. The carbon nanoparticle as claimed in claim 1, which has a surface zeta potential ranging from −20.0 mV to −39.0 mV.

5. The carbon nanoparticle as claimed in claim 1, wherein a weight ratio of the sodium alginate to the diamine ranges from 1:0.1 to 1:0.5.

6. A method for alleviating ocular angiogenesis, comprising administering to a subject in need thereof a pharmaceutical composition containing a carbon nanoparticle as claimed in claim 1.

7. The method as claimed in claim 6, wherein the pharmaceutical composition is in a dosage form for intraocular administration or topical ophthalmic administration.

8. A method for producing a carbon nanoparticle, comprising the step of subjecting sodium alginate and a diamine to a pyrolysis treatment at a temperature ranging from 160° C. to 200° C., the diamine being a C₄-C₁₀ linear aliphatic diamine.

9. The method as claimed in claim 8, wherein the temperature is 180° C.

10. The method as claimed in claim 8, wherein a weight ratio of the sodium alginate to the diamine ranges from 1:0.1 to 1:0.5.
